(19) United States
(12) Patent Application Publication
(10) Pub. No.: US 2014/0316440 A1
(43) Pub. Date: Oct. 23, 2014

(54) MULTIPLE CLIP ENDOSCOPIC TISSUE CLIPPING SYSTEM AND DEVICE

(71) Applicant: Mark Gordon, San Juan Capistrano, CA (US)

(72) Inventor: Mark Gordon, San Juan Capistrano, CA (US)

(21) Appl. No.: 14/170,446

(22) Filed: Jan. 31, 2014

Related U.S. Application Data
(60) Provisional application No. 61/849,694, filed on Feb. 1, 2013.

(51) Int. Cl. A61B 17/128; A61B 17/10; A61B 17/00

(52) U.S. Cl. CPC A61B 17/1285; A61B 17/00234; A61B 17/10; A61B 2017/00296; A61B 2017/00004
USPC 606/142

(57) ABSTRACT

An apparatus for endoscopic clipping of a surgery site comprising: a clip deployment mechanism operatively coupled to an actuator via a hollow elongate enclosure; wherein the clip deployment mechanism houses a plurality of surgical clips exterior to the enclosure; and wherein, in response to the actuator, the clip deployment mechanism sequentially deploys the surgical clips to the surgery site.

### CROSS-REFERENCE TO RELATED APPLICATIONS

This application is based on U.S. Provisional Application No. 61/849,694, filed on Feb. 1, 2013, the contents and disclosures of which are herein incorporated by reference.

### BACKGROUND OF THE INVENTION

What is disclosed within this document is a simple, low cost and innovative endoscopic surgical tissue closing device that can be deployed via an endoscopic working channel or through a laparoscope.

The system is able to dispense multiple tissue closure clips from an endoscopic probe through the working channel of a colonoscope or endoscope. It could also operate through laparoscope or trocar.

This system is designed to provide an endoscopic clipping system that will offer the best of clipping and suturing results, in a low cost, readily deployed surgical closure system.

Minimally invasive surgery has become a commonly used method for providing surgical treatment to patients in a way that has minimal side effects, and requires minimally invasive access to portions of the body where treatment is to be directed. Minimally invasive surgical access is normally accomplished via flexible endoscope, though a rigid laparoscope, or through a trocar. All of these methods allow surgical instruments, illumination sources, and various surgical and diagnostic implements access to the surgical site.

While performing this type of surgery, it is frequently necessary to close lesions or incisions found or created during the course of the surgery. It is also frequently necessary to attach additional materials such as a hernia repair mesh, a stent for vessel repair, or other means of providing interventional therapeutic devices. The current devices in use to accomplish these procedures may be mechanical clips, mechanical staples, or using sutures that are applied by a combination of surgical implements and manual manipulation of those implements.

At this time, there are a number of clipping and stapling systems in current medical, endoscopic surgical use which can clip or staple tissue together, or attach material within a surgical site. Covidien, Olympus, and Ethicon Endo-surgery, to name a few, have a number of endoscopic clipping and stapling systems commercially available for endoscopic surgical use.

For example, one common endoscopic procedure is the colonoscopy. This procedure is commonly performed for colorectal cancer screening, and is a procedure to examine the large bowel and distal part of the small bowel. The procedure is accomplished by the use of a flexible endoscope. The endoscope has the capability to illuminate the site being examined for visual inspection, irrigate the site and provide access for surgical accessories through the working channel of the endoscope. The endoscopes used typically have a working channel size between 2.8 mm and 4.1 mm in diameter, extending the length of the endoscope.

During the procedure small growths, called polyps, are sometimes found and subsequently removed via a snare or other removal means, using a device introduced through the working channel. When the polyps are removed, there may be bleeding lesions from the removal site, or from intestinal perforation.

If the lesions are bleeding significantly, they must be closed to prevent excessive bleeding. When this situation arises, the corrective action is generally the application of a metal clip to close the lesion or perforation site. In the event of a more severe bleeding episode, or intestinal perforation, a more invasive surgical correction is required. The surgical clips that are applied are generally around 25 millimeters (mm) long, and attach to the tissue wall by means of clamping several steel wire elements engaged with the tissue around or across the lesion. The clips are intended to stay in place for a few days to a few weeks, and are then sloughed off and voided by the patient. Often times, several clips may be required, either to close one perforation, or to close multiple sites.

Clips are commonly applied via an application tool, one at a time, through the working channel of the colonoscope. The clips require a dedicated, single use deployment device to deploy them that is threaded through the working channel of the endoscope.

There are significant drawbacks to this system: if multiple clips are required, the procedure can be time consuming as the application device must be withdrawn and re-loaded with a clip for each application; the clips have a range of prices; and the clips themselves are large enough that passage of body waste can dislodge them before healing is complete. As the clips are sometimes fabricated from ferrous materials, the patient is restricted in the use of an MRI for further diagnosis.

While there are a number of clip application systems designed with multiple clips, the current state of the art has a number of limitations: the clips must be designed with a geometry that allows them to be stored inside of an elongate tubular extension, the majority of these devices rely on relative linear motion between the clip and the outer tube of the device used as a closure mechanism, and subsequently damping the clip closed around the tissue to be clipped or otherwise treated.

In surgical applications other than colonoscopy, the size of this type of clip can preclude their use and deployment in procedures such as hernia repair where the application must be completed in a small space. The mesh used for hernia repair is often retained in place by the use of bio-absorbable pins or tacks. If metal clips are used, they generally are removed from the patient after healing at the site, requiring an additional procedure.

Metal clips are commonly used during laparoscopic surgery as a ligation means to close ducts or vessels during the procedure. In this case, a single clip is deployed via clipping tool through, an endoscope working channel, a trocar or a rigid laparoscope. In the application of the clip to the vessel or duct to be closed, the clip must be mechanically deformed to the closed position and the bending resistance of the metal is employed to maintain ii in the closed position. While this procedure may be reversed, the clips are generally intended to be permanent. These clips are rigid, and are predominantly made from metal such as titanium or stainless steel, and are placed one at a time, requiring the application tool to be withdrawn from the surgical site and reloaded for each clip application. The application tools are generally rigid, and require a 10 to 12 mm portal for patient site access. The current application tools are not flexible, further limiting site access.

In the case of suture application, this technique generally requires two rigid laparoscopic suture tools, and relatively larger amounts of space around the internal surgical site for the surgeon to manipulate the tools. This is a tedious process, requiring generally several minutes per suture, and requires specific suturing techniques. Suturing also has disadvantages in being technique dependent, and the sutures can damage tissue if applied with excessive tension. Sutures may be constructed of bio-resorbable and non-resorbable materials. Generally suturing has the potential for greater tissue trauma than clipping, especially in very soft tissue. It is commonly used in cases where clipping may not be relied upon to maintain positioning of the clips, where a greater tension across the tissue site is desired, or where there are spatial restrictions in the use of a clip.

It is therefore desirable to provide a multiple clip endoscopic tissue clipping system for closing incisions, lesions, attach therapeutic devices, or to close ducts or tubes. It is further desirable to provide such a system having multiple surgical located clips on the outside of a hollow elongate enclosure rather than trying to locate the clips, advancement means, and deployment apparatus all within a hollow tubular structure. Accordingly, the clips are not enclosed by the elongate enclosure, rather they are stored on the outside of the structure of the elongate enclosure, and in this way the mechanisms required to apply and advance the clips may operate independently of the clip location, the clip size and shape. This has the further advantage of allowing the advancement and deployment mechanisms adequate space and mechanical action that is independent of the clip storage method or the clip geometry. Furthermore, this arrangement allows a much greater flexibility in the design and shape of the clip. Since the clips may be larger than they would be if they shared the space of the elongate enclosure with the advancement and deployment mechanism, there is much greater latitude in shape and materials used to manufacture them. Plastic as well as metal may be used to fabricate the clip while retaining adequate strength. In addition, the clip and delivery system could be used for a drug delivery means for localized (targeted) drug delivery.

### SUMMARY OF THE INVENTION

A multiple clip endoscopic tissue clipping system is described herein that overcomes the limitations noted above.

An apparatus for endoscopic clipping of a surgery site comprises: a clip deployment mechanism operatively coupled to an actuator via a hollow elongate enclosure; wherein the clip deployment mechanism houses a plurality of surgical clips exterior to the enclosure; and wherein, in response to the actuator, the clip deployment mechanism sequentially deploys the surgical clips to the surgery site.

Other features and advantages of the present invention will become apparent from the following more detailed description, taken in conjunction with the accompanying drawings, which illustrate, by way of example, the principles of the presently described apparatus and method of its use.

### BRIEF DESCRIPTION OF THE SEVERAL VIEWS OF THE DRAWING(S)

Illustrated in the accompanying drawing(s) is at least one of the best mode embodiments of the present invention In such drawing(s):

FIG. 1 illustrates a multiple clip endoscopic tissue clipping system according to an embodiment of the present invention;

FIG. 2 illustrates an exemplary clip for use with the multiple clip endoscopic tissue clipping system according to an embodiment of the present invention;

FIGS. 3A and 3B illustrates an exemplary clip for use with the multiple clip endoscopic tissue clipping system according to an embodiment of the present invention;

FIGS. 4A and 4B illustrates an exemplary clip for use with the multiple clip endoscopic tissue clipping system according to an embodiment of the present invention;

FIG. 5 illustrates an exemplary clip for use with the multiple clip endoscopic tissue clipping system according to an embodiment of the present invention;

FIG. 6 illustrates an exemplary advancement and delivery mechanism for use with the multiple clip endoscopic tissue clipping system according to an embodiment of the present invention;

FIG. 7 illustrates an exemplary advancement and delivery mechanism for use with multiple clip endoscopic tissue clipping system according to an embodiment of the present invention;

### DETAILED DESCRIPTION OF THE PREFERRED EMBODIMENTS

The above described drawing figures illustrate the described invention and method of use in at least one of its preferred, best mode embodiment, which is further defined in detail in the following description. Those having ordinary skill in the art may be able to make alterations and modifications to what is described herein without departing from its spirit and scope. While this invention is susceptible of embodiment in many different forms, there is shown in the drawings and will herein be described in detail a preferred embodiment of the invention with the understanding that the present disclosure is to be considered as an exemplification of the principles of the invention and is not intended to limit the broad aspect of the invention to the embodiment illustrated. Therefore, it should be understood that what is illustrated is set forth only for the purposes of example and should not be taken as a limitation on the scope of the present apparatus and its method of use.

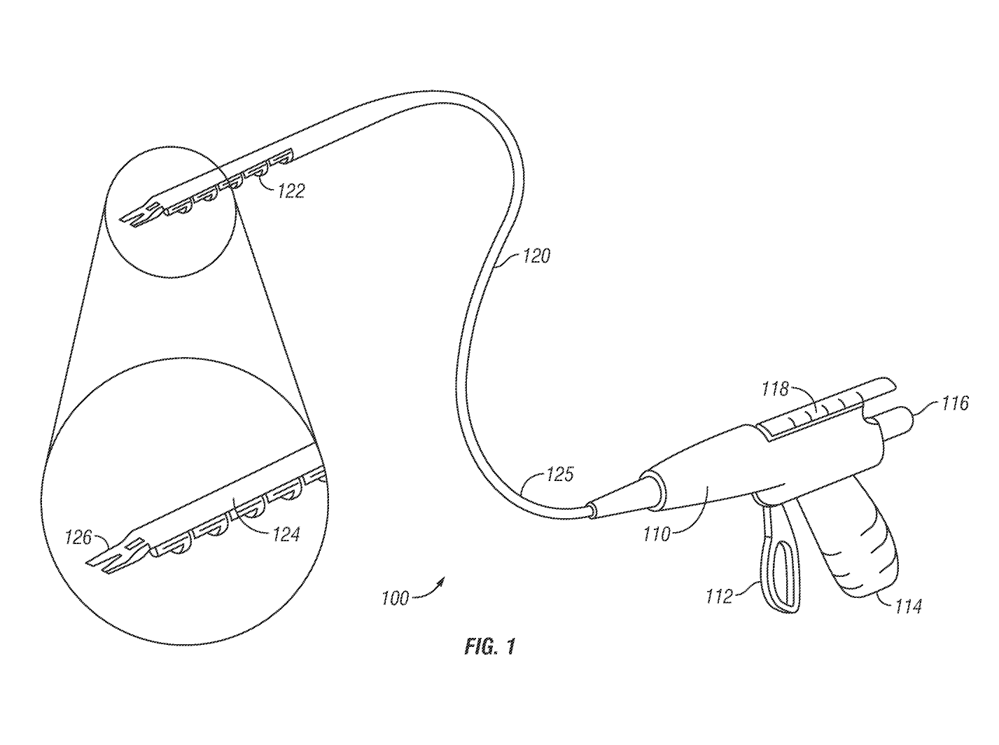

FIG. 1 illustrates an exemplary multi-clip endoscopic clipping system 100 according to at least one embodiment of the present invention. A handle assembly 110 is operatively coupled to a clip deployment mechanism 126 via a hollow elongate enclosure 120, the clip deployment mechanism housing a plurality of surgical clips 122 for use in endoscopic (or other minimally invasive) surgery substantially exterior to the enclosure. During endoscopic surgery, the clip deployment mechanism may be inserted into the surgery site and the surgical clips deployed so as to close incisions, lesions, ducts or tubes, or attach therapeutic devices, as desired. The clips are preferably arranged—for example, as in a magazine—so as to enable their sequential deployment to the surgery site without a user having to remove the deployment mechanism from the surgery site.

As illustrated in FIG. 1, the handpiece preferably includes controls for clip advancement and deployment. Accordingly, the handpiece may comprise a trigger 112 and a grip 114 for cooperatively controlling clip deployment via the clip deployment mechanism, as described further herein. Moreover, the handpiece may further comprise a clip advancer 116 for controlling the sequential advancement of clips to the deployment mechanism from a profiled member 160 (which may function as a loading area or be adjacent thereto) generally adjacent the clip deployment mechanism and housing the plurality of clips, as described further herein.

The elongate enclosure couples the handle assembly at the proximal end 125 of the enclosure to the distal end of the enclosure 124. The enclosure may be rigid or flexible, as determined by the application and type of procedure where it is to be used.

Also illustrated in FIG. 1 the plurality of clips are preferably located exterior to the elongate enclosure and are advanced as needed via the clip advancer—which may be in the form of a button that, when pressed, causes the next clip in the sequence to advance to the deployment mechanism. In some embodiments, the clip advancer is a mechanical clip advancer, such as a wire or rod housed within at least one of the handle and the elongate enclosure that transfers force applied to the button through the elongate enclosure to the clips stored in the staging area. However, the clip advancer may also include non-mechanical or electrical clip advancement components. In use, each clip is advanced sequentially into place to be deployed individually. As each clip is advanced, the magazine of clips behind the deployed clip is advanced accordingly, thus allowing the clip magazine to be advanced incrementally as clips are deployed.

According to at least one embodiment, the clips are located external to the elongate enclosure and therefore the mechanisms required to apply and advance the clips may operate independently of clip location, size and shape. It also permits the advancement and deployment mechanisms adequate space and mechanical action independent of the clip storage method or clip geometry. Additionally, since the clips may be larger than if they shared the space of the elongate enclosure with the advancement and deployment mechanism, there is much greater latitude in shape and materials used to manufacture them. Moreover, as the clips are applied by a mechanism at the distal end of the device, the practitioner will have an un-obstructed view of the clip during the application of the clip.

### Clip

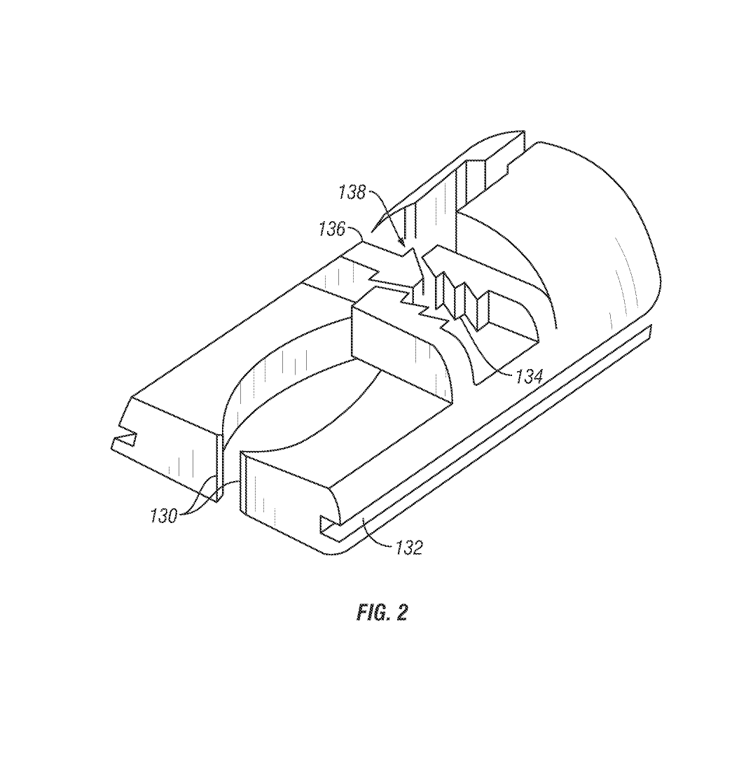

At least one embodiment for a clip for use in the multi-clip endoscopic clipping system described herein will now be described with reference to FIGS. 2-5. The clip may be fabricated by molding a thermoset polymer, metal injection molding (MIM), molding the clips from a thermoplastic polymer, or forming the clip from metal by die casting. In addition, the clip could be formed by Stereo Lithography from light cured polymer. The clip could also be formed by forging or stamping the clip from metal, though with either of these methods, secondary manufacturing operations would most likely be required. The clips may be fabricated in two (or possibly more) components that are linked together at the time of deployment, or they may be fabricated as a single component. Importantly, though exemplary methods of forming the clip are herein described, it will be understood that the clip may be formed by any other method of (or materials for) forming surgical clips that are known in the art.

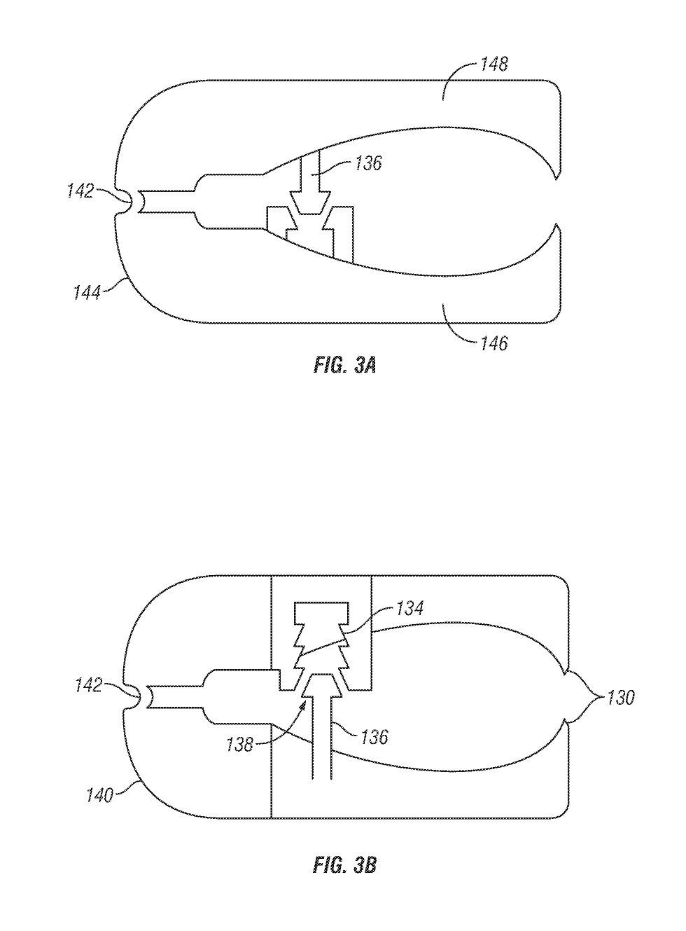

As shown in FIG. 2 and FIG. 3, the clips may be manufactured in one piece. The clip may comprise two arms 146 and 148 flexibly coupled by a connecting element 142 which acts as a flexible hinge as the two arms of the clip are opened during deployment. Preferred methods of deployment are discussed further herein.

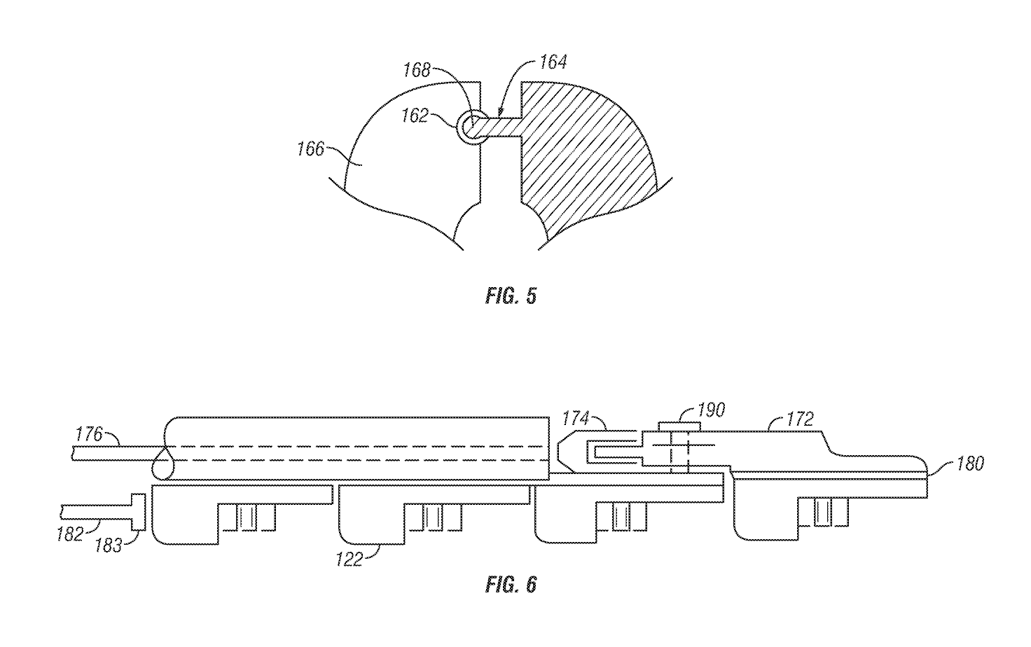

As shown in FIG. 5, the clips may be fabricated in two pieces and hingedly coupled at a connecting element 164. For example, the connecting element may comprise an appendage 168 shaped (for example as a cylinder) so as to be rotatively received by an aperture 162 shaped (for example as a negative cylindrical space) so as to rotatively receive the appendage. This permits the appendage to fit within the aperture so as to allow the clip arms to open/close when advanced on deployment jaws 172 and 192. Such two-part construction is particularly desirable for clips made of substantially more rigid material.

In some embodiments, the clips may be formed by molding them from a polymer. A bio-absorbable polymer may be used and optionally mixed with an active pharmaceutical ingredient (API). The clip may be attached to a body part, tissue or organ where it is desired to provide a controlled drug delivery by attachment of the API loaded clip to the location of interest.

As shown in FIGS. 2-5, the clips further comprise distal tips 130 operable to grasp the tissue therebetween. The distal tips may be manufactured in different configurations to provide varying degrees of tissue retention, (or grip) on the tissue. The ends may be very sharp or made with a small radius to provide a less traumatic grip on the tissue. The ends may be fabricated with multiple serrations to grasp tissue that might otherwise slip from the tips. The tips may also be fabricated to be pinched together when closed, or to have a small gap allowed, enabling various thicknesses of tissue or components to be attached/constrained.

After the clip is applied, it may be retained in place by locking the clip in place while it secures the tissue between tips. Additionally, the clip arms may be varied in cross sectional dimensions. This has the effect of varying the flexibility of the arms, thus controlling the force applied across the grasping tips.

As shown in FIG. 3B, the clip may have rounded proximal ends 140 so that the stack of clips, when lined up end to end, may flex as a group without causing the total length, referenced at the centerline to change. This feature allows the clips to be maintained in alignment while attached to the endoscope while it is in a bent or curved position. There may be a radius designed into the clips at the proximal or distal ends to accommodate the requirement for the magazine of clips to be flexible.

Referring again to FIG. 2, when the clips are fastened in place, either to tissue or to a secondary component to be attached to the patient, it is desirable that the clip remain in a closed position locking the tissue securely in place between the distal tips. Accordingly, to ensure that it is not inadvertently dislodged, the clip may further include a locking mechanism 138 operable to lock the clip in a closed position. The locking mechanism may be integral to the clip. The locking mechanism may comprise a tab portion 136 and a relief portion 134 operable to accept the tab portion and secure it therein. In at least one embodiment, the tab portion is an arrow-shaped tab and the relief portion is a series of teeth for engaging the tab in zip-tie manner. Preferably, the tab and relief is such that once the tab passes the relief, it cannot be withdrawn, thereby locking the clip in the fastened position.

In some embodiments, the clip advancement mechanism advances the clip magazine by the length of one clip. For each deployment cycle, one clip is advanced far enough in one cycle to feed one clip at a time to the deployment mechanism. The clips are arranged in their storage position so that the distal end of one clip is in contact with the proximal end of the clip preceding it.

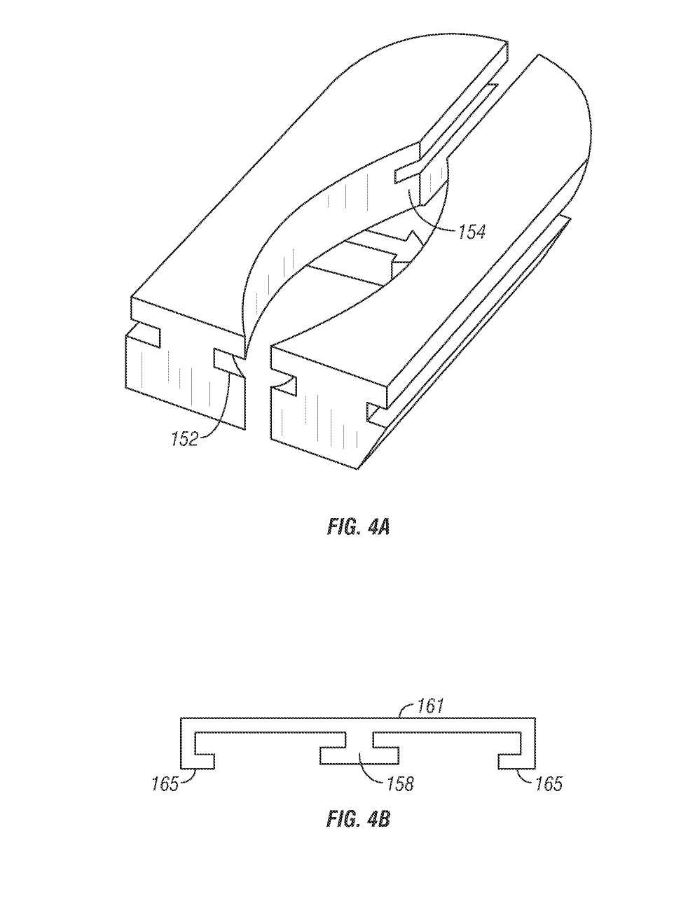

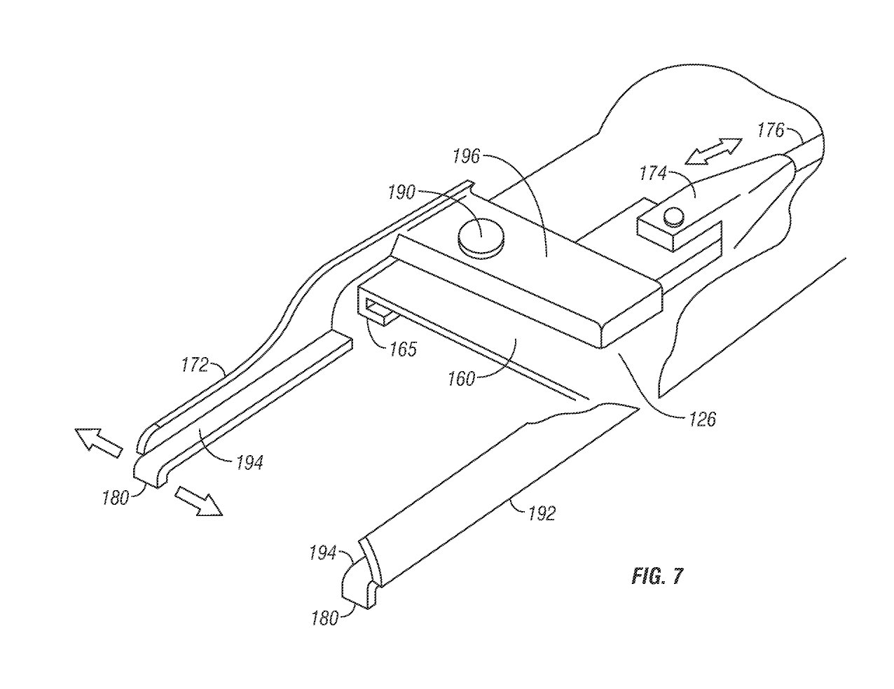

FIG. 6 and FIG. 7 illustrate a preferred embodiment wherein when the clips are attached to the elongate member, they are retained in place by a profiled member 160 attached to the elongate member—allowing them to be advanced distally on side guide rails 165, from the storage location of the profiled member to a deployment location on jaw tabs 194. Accordingly, as shown in FIG. 4, the clips comprise side grooves 132 for slidably engaging the side guide rails.

As shown in FIG. 4, in at least one embodiment, the clips are fabricated in multiple parts and guided by means of a center guide rail 158 and side guide rails. Grooves 152 and 154 are aligned with the center guide rail, and grooves 132 are aligned with the side guide rails, so as to maintain the clips in position between side rails, while still allowing the clips to be advanced when necessary. While specific exemplary embodiments are herein discussed, it should be understood by a person of ordinary skill in the art that any known method for restraining and securing surgical clips that keeps the clips in immediate proximity to the elongate member and allows them to be advanced to be engaged to the deployment mechanism may be implemented within the scope of the invention.

In at least one embodiment, the clip is compliant to tissue compression. For example, in situations where the grasped tissue compresses (or otherwise deforms), it may be desirable for the clip tips to still retain sufficient grip on the tissue. Accordingly, the clip arms may be substantially elastic so as to enable their opening to a flexed open position in response to an outward force, for grasping the tissue. Such flexion may also occur in the hinge, which may apply an elastic force to the clip arms. The degree of elasticity is preferably such that the clip tips maintain a sufficient grip on the tissue without causing injury. In operation, the elastic force may cause the clip arms to continue apply sufficient pressure on the grasped tissue to keep the clip in place should the tissue deform.

### Clip Advancement

Clip advancement according to at least one embodiment of the present invention will now be described with particular reference to FIG. 1, FIG. 6 and FIG. 7.

As shown in FIG. 1, the handle assembly houses a clip advancer—e.g. an advancement button—that may be implemented so as to advance a new clip in to the deployment mechanism. The clip may be advanced by pushing the advancement button, which may be coupled to a rod or wire 182 extending the length of the enclosure from the button to the deployment mechanism, as shown in FIG. 6. In operation, pressing the button may advance the clips distally via application of force through the wire or rod to an interface 183 adjacently engaged with the clip (or clip magazine) so as to advance the clip (or clip magazine) along the profiled member of the loading area and/or from the loading area to the deployment mechanism, shown in FIG. 7.

In some embodiments, the clips may be advanced via any mechanical, electrical, and/or fluid pressure displacement mechanisms known in the art. Preferably, the clip magazine is advanced approximately the length of one clip for each application prior to use, and is positioned on the deployment mechanism when the practitioner is ready to apply the clip.

As shown in FIG. 1, the clip advancement mechanism may also be coupled to a visual indicator 118. The visual indicator may indicate of how many clips remain. The visual indicator may be advanced by one clip indication for each clip used. The visual indicator is preferably located on the handle assembly where it is viewable by the practitioner.

### Clip Deployment

Clip deployment according to at least one embodiment of the present invention will now be described with particular reference to FIG. 1, FIG. 6 and FIG. 7.

The clip deployment mechanism preferably comprises a pair of opposing deployment jaws 172 and 192 coupled to respective lateral sides of the profiled member and extending distally therefrom. The deployment jaws may comprise deployment tabs 194 protruding interior thereto, forming side guide rails for receiving side grooves of the clips, thereby securing the clips on the deployment mechanism.

The clips may be secured to the deployment tabs by using a preferential interference fit. The deployment tabs may be slightly thicker than the clip grooves, preventing inadvertent release of the clip before the locking mechanism can be applied to lock the clip into place.

As shown in FIG. 6 and FIG. 7, the deployment mechanism preferably comprises at least one movable jaw 172, operable to open/close in response to the forward/backward movement of the handle assembly trigger. The trigger may be coupled to a wire or cable 176 that extends the length of the enclosure. The wire may terminate in a pivotal coupling to a first end of a lever 196 arranged substantially normal to the wire while in a neutral position. The pivotal coupling is preferably via a clevis or similar device. A second end of the lever may comprise a pivot pin 190 about which the lever may rotate in response to proximal/distal movement of the wire. The second end of the lever may also be affixed to the movable jaw such that rotation of the lever causes the movable jaw to open/close in response.

As shown in FIG. 1 and FIG. 7, after the clip is advanced on to the deployment mechanism, the movable jaw may be opened via forward movement of the trigger and resultant rotation of the lever. The frictional force from the inference fit of the clip grooves and the deployment tabs causes the clip arms to be opened in conjunction with the movable jaw, thereby permitting more tissue to be grasped by the clip tips. Once the desired tissue (or components attached to tissue) is engaged, the trigger may be pulled backwards (i.e. towards the grip) causing the movable jaw to close. Closure of the movable jaw exerts an inward force on the arms of the clip and causes the locking mechanism to engage, locking the tips of the clip in place tightly grasping the tissue. Returning the trigger to a neutral position causes the movable jaw to return to a neutral position. However, the locking force of the locking mechanism is such that it overcomes the frictional force of the inference fit and thereby causes the deployment tab to disengage from the clip groove, permitting the deployment mechanism to be removed from the clip.

In some embodiments, the deployment mechanism is similar in operation to a surgical forceps, and it is easily understood by practitioners skilled in the art that the present invention may be utilized with a surgical forceps function. It will also be recognized that alternative mechanical, electrical and/or other means may be utilized to open and close the clips onto the tissue without departing from the subject matter of the invention. For example, the deployment means may comprise a pair of movable jaws such that both are opened during operation, thereby increasing the span of tissue (or components attached to tissue) that may be grasped.

The clip deployment mechanism according to at least one embodiment of the present invention preferably includes one or more of the following features:

(1) the clip is securely maintained on the deployment jaws;

(2) the clip arms are opened with the opening of the deployment jaws;

(3) the clip arms/tips grasp the tissue while the clip is engaged with the deployment jaws;

(4) the deployment mechanism causes closure and locking of the clip; and

(5) the deployment jaws are able to be withdrawn once the clip is locked in place.

The enablements described in detail above are considered novel over the prior art of record and are considered critical to the operation of at least one aspect of the invention and to the achievement of the above described objectives. The words used in this specification to describe the instant embodiments are to be understood not only in the sense of their commonly defined meanings, but to include by special definition in this specification: structure, material or acts beyond the scope of the commonly defined meanings Thus if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that the named inventor believes that the claimed subject matter is what is intended to be patented.

What is claimed is:

1. An apparatus for endoscopic clipping of a surgery site comprising:
a clip deployment mechanism operatively coupled to an actuator via a hollow elongate enclosure;
wherein the clip deployment mechanism houses a plurality of surgical clips exterior to the enclosure; and
wherein, in response to the actuator, the clip deployment mechanism sequentially deploys the surgical clips to the surgery site.

2. The apparatus of claim 1,
wherein the clip deployment mechanism comprises at least one movable deployment jaw engaging one surgical clip, and
wherein the one surgical clip is openable for grasping tissue at the surgical site in response to an opening of the movable deployment jaw.

3. The apparatus of claim 2,
wherein the one surgical clip is closable to grasp tissue at the surgical site in response to a closing of the movable deployment jaw, and
wherein the clip further comprises a locking mechanism that locks the clip in a grasping orientation in response to the closing of the movable deployment jaw.

4. The apparatus of claim 1, wherein the clips are housed in a loading area exterior to and adjacent the enclosure, and are advanced in single-file to from the loading area to engage at least one deployment jaw of the clip deployment mechanism.

5. The apparatus of claim 1, wherein the clips are formed of at least one of: a bio-absorbable polymer and an active pharmaceutical agent.

6. The apparatus of claim 1, wherein the clips are formed from at least one of: a non-bio-absorbable metal and a non-bio-absorbable polymer.

7. The apparatus of claim 1, wherein the clips further comprise arms for elastically grasping tissue at the surgery site.
